(19) United States
(12) Patent Application Publication
(10) Pub. No.: US 2026/0090961 A1
(43) Pub. Date: Apr. 2, 2026

(54) ANTIPERSPIRANT CREAM

(71) Applicant: Lume Deodorant, LLC, New York, NY (US)

(72) Inventors: Shannon Klingman, New York, NY (US); Stephanie Richmond, New York, NY (US); Rebecca Reynandez, New York, NY (US); Nikita Bhatnagar, New York, NY (US)

(73) Assignee: Lume Deodorant, LLC, New York, NY (US)

(21) Appl. No.: 18/900,122

(22) Filed: Sep. 27, 2024

(51) Int. Cl. A61K 8/02; A61Q 15/00

(52) U.S. Cl. CPC A61K 8/0204; A61Q 15/00; A61K 2800/592; A61K 2800/805

(57) ABSTRACT

The present disclosure relates to antiperspirant cream composition that contains water, an antiperspirant material, an alpha-hydroxycarboxylic acid, an oil agent, a structuring agent and optionally caffeine or a xanthine derivative that has vasoconstrictive effects. The antiperspirant cream composition may be applied to a human's external skin in need or odor and perspiration control such as the user's axilla, pudendum, feet, chest, abdomen, thigh, and buttock.

### FIELD OF THE INVENTION

The present invention relates to antiperspirant compositions in cream form that provide good antiperspirant efficacy and odor control. The cream compositions exhibit excellent cosmetics and aesthetics properties and good composition stability over time. The cream compositions may be applied to any external surface of a body to control perspiration and odor at the site of application. Application sites include, but are not limited to, axilla, pudendum, feet, chest, abdomen, thigh, and buttock. The present invention further relates to methods for preparing the cream and methods of using the cream.

### BACKGROUND

Compositions designed to stop or reduce the flow of perspiration are well known in the cosmetic and chemical literature. Antiperspirants typically contain an astringent material, such as an astringent aluminum or zirconium salt. These compositions are designed to deliver the astringent material to the skin in an effective form, while being cosmetically acceptable to the user. Examples of such compositions are described for example in U.S. Pat. No. 5,019,375 and U.S. Patent Application Publication Nos. 2003/0138389; 2013/0280175; 2017/0172894; 2020/0022892; 2022/0287934 and 2023/0363992.

Topical compositions that are useful in controlling body odors by adjusting the skin's microbiome with the use of an alpha-hydroxycarboxylic acid such as mandelic acid are described in U.S. Pat. Nos. 9,668,948 and 9,566,223 and U.S. Patent Application Publication No. 2022/0071878, which are incorporated herein by reference. Topical odor controlling creams containing mandelic acid are also commercially available under the trademarks LUME® and MANDO®.

Cream compositions present particular formulation and use problems. For example, some cream compositions may be sticky and produce aesthetically undesirable levels of white chalky residue on the skin after use. Creams may also be messy and otherwise difficult to apply. Creams also present unique stability issues because the ingredients may interact causing a decrease in efficacy or undesirable product odors. In addition, the ingredients may agglomerate and/or separate from the cream resulting in a product with unacceptable homogeneity over time.

Although numerous antiperspirant and deodorants are described in the art, there always remains a need to develop an improved upon these compositions.

### SUMMARY OF THE INVENTION

The present invention is a topical antiperspirant cream composition comprising: (i) water, (ii) one or more antiperspirant materials; (iii) one or more alpha-hydroxycarboxylic acids; and (iv) one or more cosmetically acceptable auxiliary substances. The cosmetically acceptable auxiliary substances include, oil agents (sometimes referred to as emollient); structuring agents (sometimes referred to as thickeners or viscosity enhancing agents); skin conditioning agents (sometimes referred to as humectants and/or moisturizers), preservative agents, antioxidants agents, pH adjusting agents, buffering agents, chelating agents, fragrance agents and combinations of the foregoing.

In certain embodiments, the antiperspirant cream composition comprises: (i) about 40 wt % to about 70 wt % water; (ii) about 5 wt % to about 30 wt % of one or more antiperspirant materials; (iii) about 1 wt % to about 10 wt % of one or more alpha-hydroxycarboxylic acids; and (iv) one or more cosmetically acceptable auxiliary substances. In certain aspects of this embodiment, the antiperspirant cream composition may further comprise about 0.1 wt % to about 10 wt % of caffeine or a xanthine derivative that has vasoconstrictive effects.

In certain embodiments, the antiperspirant cream composition comprises: (i) about 40 wt % to about 70 wt % water; (ii) about 5 wt % to about 30 wt % of one or more antiperspirant materials; (iii) about 1 wt % to about 10 wt % of one or more alpha-hydroxycarboxylic acids; (iv) about 0.5 wt % to about 40 wt % of one or more oil agents; (v) about 0.5 wt % to about 20 wt % of one or more structuring agents; (vi) one or more skin conditioning agents and (vii) one or more additional cosmetically acceptable auxiliary substances selected from the group consisting of preservative agents, antioxidants agents, pH adjusting agents, buffering agents, chelating agents, fragrance agents and combinations of the foregoing. In certain aspects of this embodiment, the antiperspirant cream composition may further comprise about 0.1 wt % to about 10 wt % of caffeine or a xanthine derivative that has vasoconstrictive effects.

The antiperspirant cream composition may be applied to various skin areas of a human user or subject in need of antiperspirant and/or odor control such as the axilla, pudendum, feet, chest, abdomen, thigh, and buttock. The antiperspirant cream composition may be applied to the desired skin area once or more times a day. Alternatively, the antiperspirant cream composition may be applied once every 24 to 72 hours or any interval therefore. Preferably, the antiperspirant cream composition is applied shortly after showering or bathing, i.e. between about 1 to 60 minutes after showering or bathing, to the dry desired skin area. In certain aspects, the antiperspirant cream composition may be applied in the evening, preferably about 1 to 60 minutes before the user goes to bed or sleep.

The antiperspirant cream composition is prepared by mixing the ingredient in a suitable mixing vessel, preferably at a temperature between about 20° C. to about 100° C. In certain embodiments the antiperspirant cream is prepared by: (a) mixing water and one or more alpha-hydroxycarboxylic acids together to dissolve the one or more alpha-hydroxycarboxylic acids in a first mixing vessel with or without one or more cosmetic auxiliary agents; (b) mixing the one or more oil agents and one or more cosmetic auxiliary agents in a second mixing vessel; (c) combining the contents of the first and second mixing vessel; and (d) adding and mixing the one or more antiperspirant materials to the combined product of step (c). In an alternative embodiment the antiperspirant cream is prepared by: (a) mixing the one or more oil agents, one or more alpha-hydroxycarboxylic acids and one or more cosmetic auxiliary agents in a mixing vessel; (b) adding water or water that has been mixed with one or more cosmetic auxiliary agents to the mixing vessel of step (a); and (c) adding and mixing the one or more antiperspirant materials to the combined product of step (b).

### DETAILED DESCRIPTION OF THE INVENTION

Before the present invention is further described, it is to be understood that this invention is not limited to the particular embodiments described. It is also to be understood that the terminology used herein is for describing particular embodiments only, and is not intended to be limiting.

It should be noted that as used herein, the singular forms “a,” “an,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are encompassed within the invention, subject to any specifically excluded limit in the stated range. Where the stated range includes one or both of the limits, ranges excluding either or both of those included limits are also included in the invention.

As used herein the term “substantially free” means the composition contains 2 wt % or less of the stated ingredient, 1.5 wt % or less of the stated ingredient, 1 wt % or less of the stated ingredient, or 0.5 wt % of the stated ingredient depending upon the context of the description.

The term “free of” means the composition is free of detectable amounts of the stated ingredient.

The term “weight %” and “wt %” are used interchangeably and unless otherwise stated are based on the total weight of the final antiperspirant cream composition.

The present invention is an antiperspirant composition in cream form that provides good antiperspirant efficacy and odor control. In the broadest embodiment, the antiperspirant cream composition comprise: (i) water, (ii) one or more antiperspirant materials; (iii) one or more alpha-hydroxycarboxylic acids; (iv) one or more cosmetically acceptable auxiliary substances and (v) optionally caffeine or a xanthine derivative that has vasoconstrictive effects. The cosmetically acceptable auxiliary substances include, oil agents (sometimes referred to as emollient); structuring agents (sometimes referred to as thickeners or viscosity enhancing agents); skin conditioning agents (sometimes referred to as humectants and/or moisturizers), preservative agents, antioxidants agents, pH adjusting agents, buffering agents, chelating agents, fragrance agents and combinations of the foregoing.

In certain aspects of the broadest embodiment, the antiperspirant cream composition comprises: (i) about 40 wt % to about 70 wt % water, preferably about 45 wt % to about 65 wt % water and more preferably about 50 wt % to about 60 wt % water; (ii) about 5 wt % to about 30 wt % of one or more antiperspirant materials, preferably about 7.5 wt % to about 27.5 wt % of one or more antiperspirant materials and more preferably about 10 wt % to about 25 wt % of one or more antiperspirant materials; (iii) about 1 wt % to about 10 wt % of one or more alpha-hydroxycarboxylic acids, preferably about 2 wt % to about 7.5 wt % of one or more alpha-hydroxycarboxylic acids and more preferably about 2.5 wt % to about 5 wt % of one or more alpha-hydroxycarboxylic acids; (iv) about 0.5 wt % to about 40 wt % of one or more oil agents, preferably about 1 wt % to about 35 wt % of one or more oil agents and more preferably about 2.5 wt % to about 30 wt % of one or more oil agents; (v) about 0.5 wt % to about 20 wt % of one or more structuring agents, preferably about 1 wt % to about 17.5 wt % of one or more structuring agents and more preferably about 2 wt % to about 15 wt % of one or more structuring agents; (vi) 0 wt % to about 10 wt % of caffeine or a xanthine derivative that has vasoconstrictive effects, preferably about 0.1 wt % to about 7.5 wt % of caffeine or a xanthine derivative that has vasoconstrictive effects and more preferably about 0.5 wt % to about 5 wt % of caffeine or a xanthine derivative that has vasoconstrictive effects; (vii) one or more skin conditioning agents; and (viii) one or more additional cosmetically acceptable auxiliary substances selected from the group consisting of preservative agents, antioxidants agents, pH adjusting agents, buffering agents, chelating agents, fragrance agents and combinations of the foregoing.

Some embodiments of the foregoing antiperspirant cream compositions, the composition is free or substantially free of C₁-C₄ mono-alcohols such as methanol, ethanol, and isopropyl alcohol.

Some embodiments of the foregoing antiperspirant cream compositions, the composition is free or substantially free of silicone.

The antiperspirant cream compositions should exhibit a viscosity at 25° C. of about 100 cps to about 500,000 cps, about 200 cps to about 400,000 cps, about 300 cps to about 300,000 cps, about 400 cps to about 200,000 cps or about 500 cps to about 150,000 cps. The viscosity may be measured by any means commonly used in the industry. Examples of instruments and procedures for measuring the viscosity can be found in U.S. Patent Application Publication No. 2016/0128929, which is incorporated herein by reference.

The antiperspirant cream compositions also exhibit a pH of about 2, 2.5, 3, 3.5, 4, 4.5, 5, 5.5, 6, 6.5, 7 or any range within the aforementioned values. In certain embodiments the pH of the antiperspirant cream will range from 2.5 to about 6 and preferably about 2.7 to about 5.5 and more preferably about 3 to about 5. The pH may be determined by any means commonly used in the industry including the use of a pH meter or commercially available pH paper, examples of which are provided in U.S. Patent Application Publication No. 2022/0071878, which is incorporated herein by reference.

Examples of the one or more antiperspirant materials that may be used in the antiperspirant cream compositions of the present invention include but are not limited to, inorganic and organic salts of aluminum, zirconium and zinc or any mixtures of these salts. Preferred antiperspirant materials are selected from aluminum chlorohydrate, in particular aluminum chlorohydrate with the general formula [Al₂(OH)₅Cl·1-6H₂O]ₙ, preferably [Al₂(OH)₅Cl·2-3H₂O]ₙ, which can be present in non-activated or in activated (depolymerized) form, and aluminum chlorohydrate with the general formula [Al₂(OH)₄Cl₂·1-6H₂O]ₙ, preferably [Al₂(OH)₄Cl₂·2-3H₂O]ₙ, which can be present in non-activated or in activated (depolymerized) form. Also preferred are aluminum sesquichlorohydrate, aluminum dichlorohydrate, aluminum chlorohydrex propylene glycol (PG) or aluminum chlorohydrex polyethylene glycol (PEG), aluminum or aluminum zirconium glycol complexes, e.g. aluminum or aluminum zirconium propylene glycol complexes, aluminum sesquichlorohydrex PG or aluminum sesquichlorohydrex PEG, aluminum PG dichlorohydrex or aluminum PEG dichlorohydrex, aluminum hydroxide, also selected from the aluminum zirconium chlorohydrates, such as aluminum zirconium trichlorohydrate, aluminum zirconium tetrachlorohydrate, aluminum zirconium pentachlorohydrate, aluminum zirconium octachlorohydrate, the aluminum zirconium chlorohydrate glycine complexes, such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAl(SO₄)₂·12H₂O, alum), dehydrated alum (KAl(SO₄)₂ with zero to 11 moles of water of crystallization), aluminum undecylenoyl collagen amino acid, sodium aluminum lactate+aluminum sulfate, sodium aluminum chlorohydroxyacetone, aluminum bromohydrate, aluminum chloride, the aluminum salts of lipoamino acids, aluminum sulfate, aluminum lactate, aluminum chlorohydroxyallantoinate, sodium aluminum chlorohydroxyacetone, zinc chloride, zinc sulfocarbamate, zinc sulfate, zirconyl oxyhalides, in particular zirconyl oxychlorides, zirconyl hydroxyhalides, in particular zirconyl hydroxychlorides (zirconium chlorohydrate). The antiperspirant material can be present both in dissolved forms and in solubilized forms. Further examples of the antiperspirant material can be found in U.S. Patent Application Publication No. 2013/0280175, which is incorporated herein by reference.

The antiperspirant materials may be in solid or liquid forms when used in the manufacturing process to prepare the final antiperspirant cream compositions. It is preferred the antiperspirant materials is in a solid form when used in the manufacturing process to prepare the final antiperspirant cream compositions. The antiperspirant materials in a solid form have shown to improve the stability of the antiperspirant cream compositions of the present invention during manufacturing and over time such as storage in an appropriate container at 25° C. for a period of 1, 2, 3, 4, 5, 6, 7, 8, 9, 10, 11, 12, 13, 14, 15, 16, 17, 18, 19, 20, 21, 22, 23, 24 months or longer. The stability aspects of the antiperspirant cream compositions of the present invention include the antiperspirant cream composition maintaining its homogeneity, pH and/or viscosity for the desired shelf life and use. In certain aspects the pH and viscosity of antiperspirant cream compositions of the present invention do not change by more than 10%, 9%, 8%, 7%, 6%, 5%, 4%, 3%, 2%, or 1% from the initial values obtained when the antiperspirant cream composition is initial manufactured and over the shelf life or storage of the antiperspirant cream composition. For example if the initial pH of the antiperspirant cream composition is 3, after 6, 12, 18 or 24 months the pH should between 2.7 and 3.3 (a 10% change) or between 2.85 and 3.15 (a 5% change). In addition, the homogeneity of the antiperspirant cream composition should remain visibly the same over the shelf life or storage of the antiperspirant cream composition.

The amount of the one or more antiperspirant materials present in the antiperspirant cream compositions can be about 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, 15.5 wt %, 16 wt %, 16.5 wt %, 17 wt %, 17.5 wt %, 18 wt %, 18.5 wt %, 19 wt %, 19.5 wt %, 20 wt %, 20.5 wt %, 21 wt %, 21.5 wt %, 22 wt %, 22.5 wt %, 23 wt %, 23.5 wt %, 24 wt %, 24.5 wt %, 25 wt %, 25.5 wt %, 26 wt %, 26.5 wt %, 27 wt %, 27.5 wt %, 28 wt %, 28.5 wt %, 29 wt %, 29.5 wt %, 30 wt % or any range encompassed by the forgoing values. The amount of the antiperspirant materials present in the antiperspirant cream compositions should be kept below about 23 wt % to avoid unacceptable and/or undesirable chalky finishes or residue upon application of the antiperspirant cream compositions to the desired area of the user's body.

Examples of the one or more alpha-hydroxycarboxylic acids that may be used in the antiperspirant cream compositions of the present invention include, but are not limited to, glycolic acid, mandelic acid, lactic acid, citric acid and combinations thereof. The amount of the one or more alpha-hydroxycarboxylic acids present in the antiperspirant cream compositions can be about 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt % or any range encompassed by the forgoing values.

Examples of the one or more oil agents that may be used in the antiperspirant cream compositions of the present invention include, but are not limited to, hydrocarbons, oils and fats, waxes, ester oils, higher alcohols, higher fatty acids, silicone oils and combinations thereof.

Examples of the hydrocarbons that may be used in the antiperspirant cream compositions of the present invention include, but are not limited to, paraffin, ceresin, isoparaffin, hard fat, microcrystalline wax, polybutene, a polyethylene powder, liquid paraffin, squalane, petroleum jelly (vaseline), gelled hydrocarbon (Plastibase or the like), ozokerite, α-olefin oligomer, light liquid paraffin and combinations thereof.

Examples of the oils and fats that may be used in the antiperspirant cream compositions of the present invention include, but are not limited to, hard (i.e., solid or semi-solid) and soft (i.e. liquid) oils such as hydrogenated palm oil, hydrogenated rice bran oil; hydrogenated castor oil; avocado oil, linseed oil, camellia oil, macadamia nut oil, corn oil, olive oil, safflower oil, apricot kernel oil, cinnamon oil, jojoba oil, grapeseed oil, sunflower seed oil, almond oil, camellia oleifera seed oil, canola oil, sesame oil, cacao butter (cacao seed butter), coconut oil, hardened coconut oil, palm oil, palm kernel oil, Japan wax kernel oil, Japan wax, wheat germ oil, rice germ oil, rice bran oil, cottonseed oil, soybean oil, peanut oil, gold tea oil, evening primrose oil, candlenut oil, hazelnut oil, shea butter, and combinations thereof.

Examples of the waxes that may be used in the antiperspirant cream compositions of the present invention include, but are not limited to, candelilla wax, rice bran wax, cotton wax, carnauba wax, lanolin, lanolin fatty acid isopropyl ester, POE lanolin alcohol ether, POE lanolin alcohol acetate, lanolin fatty acid polyethylene glycol ester, POE hydrogenated lanolin alcohol ether, shellac wax, beeswax and combinations thereof.

Examples of the ester oil that may be used in the antiperspirant cream compositions of the present invention include, but are not limited to, isopropyl myristate, cetyl octanoate, octyl dodecyl myristate, isopropyl palmitate, isopropyl isostearate, octyl palmitate, octyl isopalmitate, cetyl palmitate, butyl stearate, isoamyl laurate, hexyl laurate, myristyl myristate, decyl oleate, isodecyl oleate, hexyl decyl dimethyl octanoate, diisopropyl sebacate, di-2-ethyl hexyl sebacate, 2-hexyl decyl myristate, 2-hexyl decyl palmitate, diisopropyl adipate, 2-hexyl decyl adipate, isononyl isononanoate, isotridecyl isononanoate, cetyl lactate, myristyl lactate, lanolin acetate, isocetyl stearate, isocetyl isostearate, isostearyl isostearate, 12-hydroxycholesteryl stearylate, cholesteryl stearate, cholesteryl oleate, phytosteryl macadamia nut fatty acid ester, phytosteryl oleate, dextrin palmitate, inulin stearate, hydrogenated jojoba oil, ethylene glycol di-2-ethyl hexylate, cetyl 2-ethyl hexanoate, dipentaerythritol fatty acid ester, N-alkyl glycol monoisostearate, neopentyl glycol dicaprate, diisostearyl malate, glyceryl di-2-heptyl undecanoate, tri-2-ethyl hexyl trimellitate, tritridecyl trimellitate, trimethylol propane tri-2-ethyl hexylate, trimethylol propane triisostearate, pentaerythritol tetra-2-ethyl hexanoate, glyceryl tri-2-ethyl hexanoate, trimethylol propane triisostearate, cetyl 2-ethyl hexanoate, glyceryl trimyristate, glyceryl tri (caprylate/caprate), glyceryl tri (caprylate/caprate/myristate/stearate), glyceride tri-2-heptyl undecanoate, castor oil fatty acid methyl ester, oleyl oleate, 2-heptyl undecyl palmitate, diisobutyl adipate, di(phytosteryl/octyl dodecyl) lauroyl glutamate, di(octyl dodecyl/phytosteryl/behenyl) lauroyl glutamate, di-2-heptyl undecyl adipate, ethyl laurate, bisethoxydiglycol cyclohexane-1,4-dicarboxylate, 2-ethyl hexyl succinate, diethoxyethyl succinate, triethyl citrate, decaglyceryl (cicosadioate/tetradecanedioate), ethyl acetate, butyl acetate, amyl acetate, triethyl citrate, (phytosteryl/isostearyl/cetyl/stearyl/behenyl) dimer dilinoleate, polyglyceryl-2 triisostearate, dimer dilinoleyl dimer dilinoleate, dipentaerythrityl dipolyhydroxystearate, glyceryl tri (behenate/isostearate/eicosadioate), polyglyceryl-10 (cicosadioate/tetradecanedioate), bisethoxydiglycol cyclohexane dicarboxylate, bis(triethylene glycol monoethyl ether) 1,4-cyclohexane dicarboxylate, bis(diethylene glycol monoethyl ether) adipate, bis(triethylene glycol monoethyl ether) adipate, diethoxyethyl succinate, diethyl hexyl succinate and combinations thereof.

Examples of the higher alcohols that may be used in the antiperspirant cream compositions of the present invention include, but are not limited to, higher alcohols having 12 to 22 carbon atoms, sterols, and the like. Preferably, the higher alcohols are saturated linear alcohols (a solid at a normal temperature) such as lauryl alcohol, myristyl alcohol, cetyl alcohol, cetostearyl alcohol, stearyl alcohol, arachidyl alcohol, and behenyl alcohol; unsaturated alcohols (a liquid at a normal temperature) such as oleyl alcohol and selachyl alcohol; branched alcohols (a liquid at a normal temperature) such as hexyl decanol, isostearyl alcohol, octyl dodecanol, decyl tetradecanol, lanolin alcohol, and isostearyl glyceryl ether; and sterols such as phytosterol and cholesterol. Any combination of the foregoing higher alcohols may also be used in the antiperspirant cream compositions of the present invention.

Examples of the higher fatty acids that may be used in the antiperspirant cream compositions of the present invention include, but are not limited to, saturated or unsaturated linear or branched fatty acids having 12 to 22 carbon atoms. For example, a saturated linear fatty acid (a solid at a normal temperature) such as a lauric acid, a myristic acid, a palmitic acid, a stearic acid, a margaric acid, an arachidonic acid, and a behenic acid; an unsaturated fatty acid (a liquid at a normal temperature) such as an oleic acid, a linoleic acid, a linolenic acid, an arachidonic acid, a palmitoleic acid, an eicosapentaenoic acid, a vaccenic acid, and a docosahexaenoic acid; or a branched fatty acid such as a lanolin fatty acid may be present. Any combination of the foregoing higher fatty acids may also be used in the antiperspirant cream compositions of the present invention.

Examples of the silicone oil that may be used in the antiperspirant cream compositions of the present invention include, but are not limited to, siloxanes such as methyl polysiloxane, methyl phenyl polysiloxane, decamethyl cyclopentasiloxane, methyl cyclopentasiloxane, high polymeric methyl polysiloxane, octamethyl cyclotetrasiloxane, methyl hydrogen polysiloxane, methyl trimethicone, dimethiconol, and a dimethiconol cross-polymer; alkyl-modified silicone such as caprylyl methicone, amino-modified silicone such as aminopropyl dimethicone and amodimethicone, cross-linked methyl polysiloxane, cross-linked alkyl-modified silicone, amino-modified silicone, polyether-modified silicone, polyglycerin-modified silicone, cross-linked polyether-modified silicone, cross-linked alkyl polyether-modified silicone, siliconealkyl chain-co-modified polyether-modified silicone, siliconealkyl chain-co-modified polyglycerin-modified silicone, polyether-modified branched silicone, polyglycerin-modified branched silicone, acrylic silicone, phenyl-modified silicone, and combinations thereof.

In certain embodiments the antiperspirant cream compositions of the present invention comprises a mixture of oil agents comprising: (a) one or more oils or fats, (b) one more ester oils and (c) one or more higher alcohols. Preferably, the oil or fats are hard oils, i.e., oils or fats that are a solid or semi-solid at temperatures of about 25° C. to about 40° C., such as hydrogenated palm oil, hydrogenated castor oil, hardened coconut oil, cocoa butter, and shea butter, and/or liquid oils or fats, i.e., oils and fats that are a liquid at temperatures of 25° C. or lower such as vegetable oils, i.e., avocado oil, corn oil, olive oil, safflower oil, apricot kernel oil, grapeseed oil, sunflower seed oil, almond oil, canola oil, sesame oil, soybean oil, peanut oil and combinations thereof. Preferably, the ester oils are stearates, myristates, laurates and palmitates such as isopropyl myristate, isopropyl laurate, isopropyl palmitate, isoamyl laurate, hexyl laurate, triglycerides such as caprylic/capric triglycerides and combinations thereof. Preferably, the higher alcohols are saturated linear alcohols such as lauryl alcohol, myristyl alcohol, cetyl alcohol, cetostearyl alcohol, stearyl alcohol, arachidyl alcohol, behenyl alcohol and combinations thereof.

The amount of the one or more oil agents present in the antiperspirant cream compositions can be about 0.5 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt % or any range encompassed by the forgoing values.

In certain aspects of the present invention, the antiperspirant cream compositions will comprise about 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt % or any range encompassed by the forgoing values of one or more hard oils or fats.

In certain aspects of the present invention, the antiperspirant cream compositions will comprise about 0.05 wt %, 0.07 wt %, 0.1 wt %, 0.12 wt %, 0.15 wt %, 0.17 wt %, 0.2 wt %, 0.22 wt %, 0.25 wt %, 0.27 wt %, 0.3 wt %, 0.32 wt %, 0.35 wt %, 0.37 wt %, 0.4 wt %, 0.42 wt %, 0.45 wt %, 0.47 wt %, 0.5 wt %, 0.52 wt %, 0.57 wt %, 0.6 wt %, 0.62 wt %, 0.65 wt %, wt %, 0.67 wt %, 0.7 wt %, 0.72 wt %, 0.75 wt %, 0.77 wt %, 0.8 wt %, 0.82 wt %, 0.85 wt %, 0.87 wt %, 0.9 wt %, 0.92 wt %, 0.95 wt %, 0.97 wt %, 1 wt %, 1.2 wt %, 1.5 wt %, 1.7 wt %, 2 wt %, 2.2 wt %, 2.5 wt %, 2.7 wt %, 3 wt %, 3.2 wt %, 3.5 wt %, 3.7 wt %, 4 wt %, 4.2 wt %, 4.5 wt %, 4.7 wt %, 5 wt % or any range encompassed by the forgoing values of one or more liquid oils or fats.

In certain aspects of the present invention, the antiperspirant cream compositions will comprise about 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt % or any range encompassed by the forgoing values of one or more ester oils.

In certain aspects of the present invention, the antiperspirant cream compositions will comprise about 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt % or any range encompassed by the forgoing values of one or more higher alcohols.

Examples of the one or more structuring agents that may be used in the antiperspirant cream compositions of the present invention include, but not limited, starches such as native starches, modified starches, cold-water soluble starches, and the like, including but not limited to corn starch, tapioca starch, potato starch, cassava starch, arrowroot starch (Maranta arundinacea root), wheat flour, sago, modified starches such as hydroxypropyl starch phosphate and cationic starches, i.e., cationic corn starch, etc. The structuring agents may also include gums such as xanthan gum, guar gum, Sclerotium gum, locust bean gum, acacia gum, konjac gels, alginin and its derivatives, namely, alginic acid, sodium alginate, potassium alginate, ammonium alginate, and calcium alginate, and the like. Polysaccharide gums and other polysaccharide thickeners may be used such as pullulan, pectin, agar, gelatin, and carrageenan (both kappa and iota forms) can be considered. Cellulose derivatives may also be used such as cellulose ether derivatives such as carboxymethyl cellulose, methyl cellulose, ethyl cellulose, ethylmethyl cellulose, hydroxyethyl cellulose, hydroxymethyl cellulose, hydroxyethylmethyl cellulose, hydroxypropyl cellulose, hydroxypropyl methyl cellulose, hydroxypropyl ethyl cellulose, hydroxyalkylcellulose polymers, and the like. Mineral agents may be used such as slurries of clay materials such as kaolin, hectorite, bentonite, laponite, silica, alumina, attapulgite, montmorillonite, hydroxyapatite, talc, etc., and mixtures or derivatives thereof. Various polymers may also be used such as polyvinyl alcohol, polyacrylic compounds such as carbomer, polylactic acid, carbomer polymers, and the like. Additional examples of structuring agent may also include silicone containing compounds, e.g., silicone oils, silicone elastomers and/or polyorganosiloxanes. Examples of the foregoing can be found in U.S. Pat. No. 10,286,030, which is incorporated herein by reference.

The amount of the one or more structuring agents present in the antiperspirant cream compositions can be about 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, 15.5 wt %, 16 wt %, 16.5 wt %, 17 wt %, 17.5 wt %, 18 wt %, 18.5 wt %, 19 wt %, 19.5 wt %, 20 wt % or any range encompassed by the forgoing values.

In certain embodiments of the present invention, the one or more structuring agents present in the antiperspirant cream composition may comprises one or more starches, such as Maranta arundinacea root, tapioca starch, corn starch, potato starch and modified starches such as hydroxypropyl starch phosphate and combinations of the foregoing.

In certain embodiments, the antiperspirant cream compositions of the present invention may also comprise one or more skin conditioning agents. As used herein skin conditioning agents include compounds that hydrate or improve the texture and feel of skin and include humectants, moisturizers and emollients. In certain embodiments, the antiperspirant cream composition will comprise about 0.0005 wt % to about 20 wt %, preferably about 0.0007 wt % to about 15 wt % and more preferably about 0.001 wt % to about 10 wt % of one or more skin conditioning agents. Examples of skin conditioning agents include, but are not limited to, glycerin, propylene glycol, butylene glycol, pentylene glycol, isopentyldiol, sodium pyrrolidine carboxylic acid (“PCA”), Carbowax 200, Carbowax 400, and Carbowax 800, PPG-15 stearyl ether, lanolin alcohol, lanolin, lanolin derivatives, cholesterol, petrolatum, isostearyl neopentanoate, octyl stearate, mineral oil, isocetyl stearate, Ceraphyl 424 (myristyl myristate), octyl dodecanol, dimethicone (Dow Corning 200-100 cps), phenyl trimethicone (Dow Corning 556), Dow Coning 1401 (cyclomethicone and dimethiconol), and cyclomethicone (Dow Corning 344), and Miglyol 840 (manufactured by Huls; propylene glycol dicaprylate/dicaprate) guanidine, urea, glycolic acid, glycolate salts (e.g. ammonium and quaternary alkyl ammonium), aloe vera in any of its variety of forms (e.g., aloe vera gel), polyhydroxy alcohols such as sorbitol, mannitol, xylitol, erythritol, glycerol, hexanetriol, butanetriol, propylene glycol, butylene glycol, hexylene glycol and the like, polyethylene glycols, sugars (e.g., melibiose), starches, sugar and starch derivatives (e.g., alkoxylated glucose, fructose, glucosamine), hyaluronic acid, lactamide monoethanolamine, acetamide monoethanolamine, panthenol, squalene, arginine, trehalose, ethylhexylglycerin, allantoin and combinations thereof. Additional examples of skin conditioning agents can be found in U.S. Pat. Nos. 5,804,203 and 8,741,357, which are incorporated herein by reference.

In certain embodiments, the antiperspirant cream compositions of the present invention may also comprise about 0.1 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt % or any range encompassed by the forgoing values of caffeine or xanthine derivatives that have vasoconstrictive effects. Examples of the xanthine derivatives are described in U.S. Patent Application Publication No. 2022/0071878, which is incorporated herein by reference.

Examples of preservatives that may be included in the antiperspirant cream compositions of the present invention include, but are not limited to, alkali metal sorbates, alkali metal benzoates, alcohols, glycols, parabens, quaternary-nitrogen containing compounds, isothiazolinones, aldehyde-releasing compounds and halogenated compounds. Specific examples of preservatives include phenoxyethanol, sodium benzoate, potassium benzoate, isopropyl alcohol, benzyl alcohol and combinations thereof.

Examples of antioxidants that may be employed in the antiperspirant cream compositions present invention include, but are not limited to, niacinamide, peptides, such as di-, tri-, tetra and hexa-peptides, acetyl cysteine, ascorbic acid polypeptide, ascorbyl dipalmitate, ascorbyl methylsilanol pectinate, ascorbyl palmitate, ascorbyl stearate, butylated hydroxyanisole (BHA), butylated hydroxytoluene (BHT), t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, Helianthus annus (sunflower) seed oil, hydroquinone, isooctyl thioglycolate, kojic acid, Laminaria digitata extract, magnesium ascorbate, magnesium ascorbyl phosphate, methylsilanol ascorbate, natural botanical anti-oxidants such as Camellia sinensis green tea extract, Nelumbo nucifera leaf, flower or germ extract, vitris vinifera (grape) callous culture extract, nordihydroguaiaretic acid, octyl gallate, phenylthioglycolic acid, potassium ascorbyl tocopheryl phosphate, potassium sulfite, propyl gallate, quinones, rosmarinic acid, Rosa moschata seed oil, Rosmarinus officinalis (rosemary) leaf extract, sodium ascorbyl phosphate, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tremella fuciformis (mushroom) extract, tris(nonylphenyl) phosphite, Zea mays (corn) oil and combinations thereof.

The one or more antioxidants are present in the antiperspirant cream compositions of the present invention in an amount of about 0.001 wt % to about 2.5 wt %, preferably about 0.0025 wt % to about 2 wt % and more preferably about 0.005 wt % to about 1.5 wt %.

Examples of pH modifying agents that may be used in the antiperspirant cream compositions of the present invention include, but are not limited to, compounds that are added to raise or low the pH of the final composition as well as compounds that are added to buffer the final antiperspirant cream composition. Compounds that may be added to raise or lower the pH of the antiperspirant cream composition include, but are not limited to, phosphoric acid and/or phosphate salts, citric acid and/or citrate salts, hydroxide salts (i.e., calcium hydroxide, sodium hydroxide, potassium hydroxide) and amines, such as triethanolamine.

Examples of buffer agents that may be used in the antiperspirant cream compositions of the present invention include, but are not limited to, acetic acid and salts, citric acid and salts, boric acid and salts, and phosphoric acid and salts such as mono- and di-potassium phosphate, citric acid/sodium citrate, and dibasic sodium phosphate/citric acid.

Examples of chelating agents that may be used in the antiperspirant cream compositions of the present invention include, but are not limited to, citric acid and salts thereof, disodium EDTA, tetrasodium EDTA, tetrasodium glutamate diacetate, trisodium nitrilotriacetate (aks trisodium NTA) and phytic acid.

Examples of fragrances that may be used in the antiperspirant cream compositions of the present invention include, but are not limited to, oils such as angelica radix oil, aniseed oil, arnica blossom oil, basil oil, bay oil, bergamot oil, champak blossom oil, fir oil, turpentine oil, elemi oil, eucalyptus oil, fennel oil, pine needle oil, galbanum oil, geranium oil, ginger grass oil, guaiac wood oil, balsam of gurjun oil, helichrysum/chasteweed oil, ho oil, ginger oil, iris oil, cajeput oil, calmus oil, chamomile oil, camphor oil, canaga oil, cardamom oil, cassia oil, fir cone oil, balsam of kopaiva oil, coriander oil, crisped mint oil, caraway oil, cumin oil, lavender oil, lemon grass oil, lime oil, tangerine oil, melissa oil, ambrette oil, myrrh oil, carnation oil, neroli oil, niaouli oil, oliban oil, orange oil, origanum oil, palmarosa oil, patchouli oil, peru balsam oil, petit grain oil, peppermint oil, pimento oil, pine oil, rose oil, rosemary oil, sandal wood oil, celery oil, lavender oil, star anise oil, turpentine oil, thuja oil, thyme oil, verbena oil, vetiver oil, juniper berry oil, absinthe oil, winter green oil, ylang-ylang oil, hyssop oil, cinnamon oil, cinnamon leaf oil, citronella oil, lemon oil, cypress oil and combinations thereof. Additional examples can be found in U.S. Patent Application Publication Nos. 2003/0138389 and 2010/0047296, which are incorporated herein by reference.

Additional specific examples of the cosmetically acceptable auxiliaries that may be used in the antiperspirant cream compositions of the present invention can be found the various U.S. Published patent applications mentioned previously as well as the CTFA International Cosmetic Ingredient Dictionary and Handbook (2008), 12^{th} Edition. The skilled artisan is aware that some of the cosmetically acceptable auxiliaries may be included in more than one of the foregoing classifications. Stated another way, some of the cosmetically acceptable auxiliaries may impart one or more properties to the antiperspirant cream composition.

The antiperspirant cream compositions of the present invention may be prepared by any method commonly known in the industry and may include blending, mixing, emulsifying, heating, cooling steps or any combination thereof. In one embodiment, the antiperspirant cream is prepared by mixing the ingredient in a suitable mixing vessel, preferably at a temperature between about 20° C. to about 100° C., preferably about 30° C. to about 100° C. and more preferably about 40° C. to about 100° C. The mixing temperature should be a temperature sufficient to keep the majority of ingredients and particularly the oil agents in a liquid state. In one embodiment the antiperspirant cream is prepared by: (a) mixing water and one or more alpha-hydroxycarboxylic acids together to dissolve the one or more alpha-hydroxycarboxylic acids in a first mixing vessel with or without one or more cosmetic auxiliary agents; (b) mixing the one or more oil agents and one or more cosmetic auxiliary agents in a second mixing vessel; (c) combining the contents of the first and second mixing vessels; and (d) adding and mixing the one or more antiperspirant materials to the combined product of step (c). In an alternative embodiment, the antiperspirant cream is prepared by: (a) mixing the one or more oil agents, one or more alpha-hydroxycarboxylic acids with or without the one or more cosmetic auxiliary agents in a mixing vessel; (b) adding water or water that has been mixed with one or more cosmetic auxiliary agents to the mixing vessel of step (a); and (c) adding and mixing the one or more antiperspirant materials to the combined product of step (b). To improve the processing of the antiperspirant cream composition it is preferred that the antiperspirant material is added in a dry or solid form during the manufacturing process. It is also preferred that the antiperspirant material is added after the water and oil agents have been mixed, and most preferably as the final additive in the manufacturing process.

The antiperspirant cream compositions of the present invention may be packaged and dispensed in any suitable container. Containers can include a bottle, a tube (metal, plastic or laminate), a pressurized container, or pouches. The containers may include amounts of the antiperspirant cream composition for single or multiple applications. Containers may include indicia on its surface that instruct the subject on its application and use. Instructions for use may also be printed separately and packaged with the container comprising the antiperspirant cream composition.

Containers comprising multiple applications of the antiperspirant cream composition can dispense a pre-determined amount of the antiperspirant cream composition via a metered or calibrated spray, pump, or squeeze mechanism. In other embodiments, the container can be squeezed (e.g., metal, laminate, or plastic tube) to dispense a desired amount of the composition.

In certain embodiments, the user or human subject in need of or desiring antiperspirant and odor control will apply the desired amount of the antiperspirant cream composition to a clean and preferably dry skin surface once every 6 to 72 hours or any interval therefore. Preferably, the antiperspirant cream composition is applied every 12 hours, 24 hours, 36 hours, 48 hours, 60 hours or 72 hours. The antiperspirant cream composition may be applied to various skin areas of a human subject in need or antiperspirant and/or odor such as the axilla, pudendum, feet, chest, abdomen, thigh, and buttock. Preferably, the antiperspirant cream composition is applied shortly after showering or bathing, i.e. between about 1 to 60 minutes after showering or bathing, to the dry desired skin area. In certain aspects, the antiperspirant cream composition may be applied in the evening, preferably about 1 to 60 minutes before the user goes to bed or sleep. In certain embodiments, the user or subject will apply about 0.1 to about 2.0 grams, preferably 0.20 grams to about 1.5 grams and more preferably, about 0.3 grams to about 1.6 grams to the target area. The amount will be adjusted depending upon the surface area to be treated.

Although any methods and materials similar or equivalent to those described herein can also be used in the practice of the present invention, the preferred methods and materials are now described. All publications mentioned herein are incorporated herein by reference to disclose and describe the methods and/or materials in connection with which the publications are cited.

The following examples are provided by way of illustration only and are by no means intended to be limiting.

### Example 1

Antiperspirant compositions in accordance with the present invention may be prepared having the following:

| | WEIGHT PERCENT | | |
|---|---|---|---|
| INGREDIENT | Preferred | More Preferred | Most Preferred |
| Water | 40-70 | 45-65 | 50-60 |
| One or more antiperspirant | 5-25 | 7.5-25 | 10-25 |
| materials | | | |
| Mandelic acid, lactic acid | 1-10 | 2-7.5 | 2.5-5 |
| or a combination thereof | | | |
| One or more starches | 0.5-20 | 1-17.5 | 2-15 |
| Caffeine | 0-10 | 0.1-7.5 | 0.5-5 |
| One or more hard | 0-10 | 0.5-9.5 | 1-9 |
| oils or fats | | | |
| One or more liquid | 0-5 | 0.05-4 | 0.1-3 |
| oils or fats | | | |
| One or more ester oils | 0-10 | 0.5-9.5 | 1-9 |
| One or more higher | 0-10 | 0.5-9.5 | 1-9 |
| alcohols | | | |

The antiperspirant cream compositions summarized in the above table may be further comprise one or more skin conditioning agents, preservative agents, antioxidants agents, pH adjusting agents, buffering agents, chelating agents, fragrance agents and combinations of the foregoing. The antiperspirant cream compositions should have a pH of about 3 to about 4.5; a viscosity of 10,000 to 150,000 at room temperature when measured with Brookfield viscometer and a shelf life of 18 to 36 months.

The antiperspirant cream compositions summarized in the above table may be prepared by any process that produces a uniform composition. In certain embodiments, water and at least one starch are mixed in a first container and heated to about 30° C. to about 65° C., preferably about 40° C. to about 60° C. The mandelic acid, lactic acid or a combination thereof, the caffeine and optionally one or more cosmetically acceptable auxiliary substances such as water soluble or water miscible skin conditioning agents, preservatives, pH adjusting agents and/or chelating agents are added to the heated aqueous mixture in the first container and mixed, preferably until the a solution is obtained. The aqueous composition/solution in the first container may be further heated to about 70° C. to about 95° C., preferably to about 80° C. to about 90° C. The one or more hard oils and fats, the one or more liquid oils or fats, the one or more ester oils and the one or more higher alcohols are added to a second container and mixed while heating to a temperature of about 70° C. to about 95° C., preferably about 80° C. to about 90° C. The heated contents of the first container and the heated contents of the second container are mixed while maintain the temperature between about 70° C. to about 95° C., preferably to about 80° C. to about 90° C. to obtain a uniform base mixture. Additional cosmetically acceptable auxiliary substances may be added to the uniform base mixture if desired. The uniform base mixture is cooled to about 35° C. to about 55° C., preferably to about 40° C. to about 50° C. and one or more antiperspirant materials are added to the cooled base mixture. Optionally one or more additional cosmetically acceptable auxiliary substances such as additional structuring agents, additional skin conditioning agents and/or fragrances may be added to the cooled base mixed prior to, concurrently with, or subsequent to the addition of the one more antiperspirant materials. The cooled base mixture with the one or more antiperspirant materials and optional cosmetically acceptable auxiliary substances is mixed until a uniform antiperspirant cream composition is obtained. The antiperspirant cream composition is then filled into a suitable package for distribution and use.

### Example 2

An antiperspirant cream compositions in accordance with the present invention was prepared having the following composition:

| Ingredient | Weight Percent |
|---|---|
| Water | 50-60 |
| Aluminum Sesquichlorohydrate | 12-17 |
| Maranta Arundinacea | 2-7 |
| Root Powder | |
| Isoamyl Laurate | 1-8 |
| Mandelic Acid | 2-6 |
| Hydropropyl Starch Phosphate | 1-6 |
| Cetearyl Alcohol/ | 1-6 |
| Cetearyl Glucoside | |
| Tapioca Starch | 0.5-5 |
| Caffeine | 0.5-3.5 |
| Caprylic/Capric | 0.5-3.5 |
| Triglyceride | |
| Hydrogenated Castor Oil | 0.5-3.5 |
| Behenyl Alcohol | 0.5-3.5 |
| Stearly Alcohol | 0.3-2 |
| Dimethicone | 0.3-2 |
| Panthenol | 0.3-2 |
| Pentylene Glycol | 0.3-2 |
| Theobroma Cacao | 0.3-2 |
| (Cocoa) Seed Butter | |
| Potassium Hydroxide | 0.3-2 |
| Tocopherol | 0.1-0.5 |
| Sodium Benzoate | 0.1-0.5 |
| Helianthus Annuus | 0.05-0.5 |
| (Sunflower) Seed Oil | |
| Allantoin | 0.01-0.1 |
| Tetrasodium Glutamate | 0.01-0.1 |
| Diacetate | |
| Aloe barbadensis | 0.0001-0.05 |
| Leaf Juice | |

The antiperspirant cream composition of this example may be prepared by any process that produces a uniform composition including the process described in Example 1.

The antiperspirant cream compositions described in the above table had a pH of about 3.5 to 4.0 and a viscosity of 15,000 to 60,000 cps at room temperature when measured with Brookfield viscometer.

The antiperspirant cream composition of this example had acceptable aesthetic properties, i.e., was smooth and not gritty, dried quickly (less than a minute after application) and left a minimal but user acceptable chalky residue. The antiperspirant cream composition of this example provide effective perspiration and odor control.

The aluminum sesquichlorohydrate employed in this example was a solid form. Attempts of use a liquid form of the aluminum sesquichlorohydrate failed to produce a stable cream.

### Example 3

An antiperspirant cream compositions in accordance with the present invention was prepared having the following composition:

| Ingredient | Weight Percent |
|---|---|
| Water | 40-50 |
| Aluminum Sesquichlorohydrate | 20-25 |
| Isopentyldiol | 3-8 |
| Isoamyl Laurate | 2-8 |
| Mandelic Acid | 2-6 |
| Hydropropyl Starch Phosphate | 2-6 |
| Cetearyl Alcohol/ | 1-6 |
| Cetearyl Glucoside | |
| Caffeine | 0.5-4 |
| Caprylic/Capric | 0.5-4 |
| Triglyceride | |
| Hydrogenated Castor Oil | 0.5-4 |
| Behenyl Alcohol | 0.5-4 |
| Stearly Alcohol | 0.3-2 |
| Dimethicone | 0.3-2 |
| Theobroma Cacao | 0.3-2 |
| (Cocoa) Seed Butter | |
| Potassium Hydroxide | 0.3-2 |
| Tocopherol | 0.1-0.5 |
| Sodium Benzoate | 0.1-0.5 |
| Helianthus Annuus | 0.05-0.5 |
| (Sunflower) Seed Oil | |
| Tetrasodium Glutamate | 0.01-0.1 |
| Diacetate | |
| Glucose | 0.0005-0.05 |
| Aloe barbadensis | 0.0001-0.05 |
| Leaf Juice | |
| Sodium Hydroxide | 0.0001-0.05 |
| Trisodium NTA | 0.00001-0.005 |

The antiperspirant cream composition of this example may be prepared by any process that produces a uniform composition including the process described in Example 1.

The antiperspirant cream compositions described in the above table had a pH of about 3.5 to 4.0 and a viscosity of 15,000 to 60,000 cps at room temperature when measured with Brookfield viscometer.

The antiperspirant cream composition of this example had acceptable aesthetic properties, i.e., was smooth and not gritty, dried quickly (less than a minute after application) and left a minimal but user acceptable chalky residue. The antiperspirant cream composition of this example provide effective perspiration and odor control.

The aluminum sesquichlorohydrate employed in this example was a solid form. Attempts of use a liquid form of the aluminum sesquichlorohydrate failed to produce a stable cream.

The invention described herein may be practiced in the absence of any element or limitation which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An antiperspirant cream composition comprising:
(i) about 40 wt % to about 70 wt % water;
(ii) about 5 wt % to about 30 wt % of one or more antiperspirant materials;
(iii) about 1 wt % to about 10 wt % of one or more alpha-hydroxycarboxylic acids;
(iv) about 0.5 wt % to about 40 wt % of one or more oil agents;
(v) about 0.5 wt % to about 20 wt % of one or more structuring agents;
(vi) 0 wt % to about 10 wt % of caffeine or a xanthine derivative that has vasoconstrictive effects;
(vii) one or more skin conditioning agents; and
(viii) one or more additional cosmetically acceptable auxiliary substances selected from the group consisting of preservative agents, antioxidants agents, pH adjusting agents, buffering agents, chelating agents, fragrance agents and combinations of the foregoing.

2. The antiperspirant cream composition of claim 1 wherein the one or more antiperspirant materials is an inorganic or organic salt of aluminum, zirconium or zinc.

3. The antiperspirant cream composition of claim 2 wherein the one or more antiperspirant materials is aluminum chlorohydrate.

4. The antiperspirant cream composition of claim 1 wherein the one or more alpha-hydroxycarboxylic acids is selected form the group consisting of mandelic acid, lactic acid and combinations thereof.

5. The antiperspirant cream of claim 1 wherein the one or more oil agents comprises:
(a) one or more hard oils or fats;
(b) one or more liquid oils or fats;
(c) one or more ester oils; and
(d) one or more higher alcohols.

6. The antiperspirant cream composition of claim 5 wherein:
(a) the one or more hard oils or fats are selected from the group consisting of hydrogenated palm oil, hydrogenated rice bran oil; hydrogenated castor oil, hardened coconut oil, cacao butter (cacao seed butter), shea butter and combinations thereof;
(b) the one or more liquid oils or fats are selected from the group consisting or avocado oil, corn oil, olive oil, safflower oil, apricot kernel oil, grapeseed oil, sunflower seed oil, almond oil, canola oil, sesame oil, coconut oil, cottonseed oil, soybean oil, peanut oil and combinations thereof;
(c) the one or more ester oils are selected from the group consisting of stearate esters, myristate esters, laurate esters, palmitate esters and combinations thereof; and
(d) the one or more higher alcohols are selected from the group consisting of lauryl alcohol, myristyl alcohol, cetyl alcohol, cetostearyl alcohol, stearyl alcohol, arachidyl alcohol, behenyl alcohol and combinations thereof.

7. The antiperspirant cream composition of claim 1 wherein the one or more structuring agents comprises at least one starch.

8. The antiperspirant cream composition of claim 7 wherein the one or more structuring agents comprises a starch selected from the group consisting of Maranta arundinacea root, tapioca starch, corn starch, potato starch, hydroxypropyl starch phosphate and combinations thereof.

9. The antiperspirant cream composition of claim 1 comprising:
(i) about 50 wt % to about 60 wt % water;
(ii) about 10 wt % to about 27.5 wt % of one or more aluminum chlorohydrate antiperspirant materials;
(iii) about 2.5 wt % to about 5 wt % of one or more alpha-hydroxycarboxylic acids selected from the group consisting of mandelic acid, lactic acid and combinations thereof;
(iv) the one or more oil agents comprises:
(a) one or more hard oils or fats selected from the group consisting of hydrogenated palm oil, hydrogenated rice bran oil; hydrogenated castor oil, hardened coconut oil, cacao butter (cacao seed butter), shea butter and combinations thereof;
(b) one or more liquid oils or fats selected from the group consisting or avocado oil, corn oil, olive oil, safflower oil, apricot kernel oil, grapeseed oil, sunflower seed oil, almond oil, canola oil, sesame oil, coconut oil, cottonseed oil, soybean oil, peanut oil and combinations thereof;
(c) one or more ester oils selected from the group consisting of stearate esters, myristate esters, laurate esters, palmitate esters and combinations thereof; and
(d) one or more higher alcohols selected from the group consisting of lauryl alcohol, myristyl alcohol, cetyl alcohol, cetostearyl alcohol, stearyl alcohol, arachidyl alcohol, behenyl alcohol and combinations thereof;
(v) the one or more structuring agents comprises a starch selected from the group consisting of Maranta arundinacea root, tapioca starch, corn starch, potato starch, hydroxypropyl starch phosphate and combinations thereof;
(vi) 0.5 wt % to about 5 wt % of caffeine.

10. A method for preparing the antiperspirant cream composition of claim 1 comprising:
(a) mixing the water and the one or more alpha-hydroxycarboxylic acids together to dissolve the one or more alpha-hydroxycarboxylic acids in a first mixing vessel;
(b) mixing the one or more oil agents and one or more cosmetic auxiliary agents in a second mixing vessel;
(c) combining the contents of the first and second mixing vessels; and
(d) adding and mixing the one or more antiperspirant materials to the combined product of step (c).

11. A method for preparing the antiperspirant cream composition of claim 1 comprising:
(a) mixing the one or more oil agents, and the one or more alpha-hydroxycarboxylic acids in a mixing vessel;
(b) adding water to the mixing vessel of step (a); and
(c) adding and mixing the one or more antiperspirant materials to the combined product of step (b).
